(19) United States
(12) Patent Application Publication
(10) Pub. No.: US 2023/0190756 A1
(43) Pub. Date: Jun. 22, 2023

(54) PHARMACEUTICAL FORMULATION FOR THE ELIMINATION OF CAUSATIVE AGENTS OF HERPESVIRAL INFECTIONS FROM THE TISSUES OF A MACROORGANISM

(71) Applicants: Boris Farber, Brooklyn, NY (US); Sof'ya Farber, Brooklyn, NY (US); ARTUR VIKTOROVICH MARTYNOV, Kharkov (UA)

(72) Inventors: Boris Farber, Brooklyn, NY (US); Sof'ya Farber, Brooklyn, NY (US); ARTUR VIKTOROVICH MARTYNOV, Kharkov (UA)

(21) Appl. No.: 16/893,903

(22) PCT Filed: Dec. 8, 2017
(86) PCT No.: PCT/RU2017/000920

§ 371 Date: Jun. 5, 2020

(51) Int. Cl. A61K 31/522; A61K 31/353; A61K 31/47; A61K 47/12; A61P 31/22; C40B 40/04

(52) U.S. Cl. CPC A61K 31/522; A61K 31/353; A61K 31/47; A61K 47/12; A61P 31/22; C40B 40/04

(57) ABSTRACT

A pharmaceutical composition is proposed for the elimination of the causative agents of herpes virus infections from macroorganism tissues, including histone deacetylase inhibitors, acyclovir derivatives, epigallocatechin gallate and glycyrrhizin, characterized in that it additionally contains a supramolecular structure of a combination of gator epigalocatechin gallate and galactate epigalocatechol at least two covalent modifying agents.

Also, the composition may include such substances as chorlecalciferol, toll receptor activators acridonoacetic acid, tilorone, zymosan, imidazoquinoline, imiquimod. As modifiers of the structures of epigalocatechin gallate and glycyrrhizin can be used: succinic anhydride, acetic anhydride, propionic anhydride, butane anhydride, acetic-propionic anhydride, acetic-buttanic anhydride, glutaric anhydride, phthalic anhydride, citric anhydride, citric anhydride citric anhydride, isolimonic anhydride, acetyl chloride, acetyl fluoride, propionyl chloride, butyroyl chloride, ethoxyoxalyl monochloride, monochloroacetic acid.

### TECHNICAL FIELD

The invention relates to pharmacy, organic and bioorganic combinatorial chemistry. Namely, to two new combinatorial libraries of derivatives of epigallocatechin and glycyrrhizin in the form of supramolecular structures, which, when used without separation into individual components in the pharmaceutical composition, have powerful antiviral properties and can lead to the elimination of pathogen viral infections caused by representatives of the genus Herpesviridae.

### PRIOR LEVEL STATE OF THE ART

Herpesvirus infection can exist in humans in various forms: lifelong latent persistence, recurrent course, the formation of immunodeficiencies, somatic immune-dependent pathology, chronic inflammation, tumor processes and can even integrate into the gene apparatus. A peculiarity of the course of herpesvirus infections is the possibility of involving many organs and systems in the infectious process. This accounts for the variety of diseases caused by herpes viruses, ranging from simple skin-mucous membranes to life-threatening generalized infections.

An important property of herpes viruses is their ability, after the initial infection of a person in childhood, to persist and reactivate for life under the influence of various exogenous and endogenous provoking factors. Herpesvirus infection is best characterized by one figurative expression: “Once infected—infected for life.” Specific antibodies that appear in response to the introduction of herpes viruses often do not provide a remedy for viruses and often do not prevent a relapse of the disease.

In this regard, practicing physicians—obstetricians, pediatricians, therapists, are primarily faced with the tasks of timely diagnosis of infection and determining the activity of its course. Correct assessment and interpretation of the results of clinical, specific laboratory, and instrumental studies allow us to develop adequate and safe medical and rehabilitation measures for infected (and sick) people, be it, a pregnant woman, fetus, child, or adult patient.

In herpes, as in other chronic diseases with viral persistence, inferiority of immune reactions often develop, due to the insufficiency of various parts of the immune system and its inability to eliminate the virus. Although persistent throughout life, at times in high concentration low-affinity virus-neutralizing antibodies may prevent the spread of viruses and do not prevent the reccurrence of viral relapses. The long-term presence of herpes viruses in human body becomes possible due to a complex strategy of confrontation and “viral escape” from the host's immune system.

To achieve this condition, three pathways of the pathogen strategy can be distinguished: Firstly, “secret presence”, which allows to virus to remain unrecognized immune system for a long time, localized in a latent state in neurons (herpes simplex virus—HSV), lymphoid cells (Epstein virus—Barr—EBV) and hematopoietic cells (cytomegalovirus—CMV). The second pathway is, “exploitation”, the use of immune responses in their own interests. Lastly the pathway of “sabotage”, damage to immune defense mechanisms. It is precisely the “sabotage” strategy that is believed to underlie the virus-induced immunosuppression that prevents the complete removal of the pathogen and, as a result, supports the chronic course of the infection. Herpes viruses are currently clearly classified and integrated into the extensive Herpesviridae family

The Herpesviridae family includes more than 100 members, 8 of which are the most pathogenic for humans (human herpes virus—HHV).

Outwardly, the similarity of herpes viruses is so great that under an electron microscope they are almost impossible to distinguish. The individuality of “relatives” begins to appear only when it comes to the antigenic properties of virion proteins and the degree of DNA homology. Herpes viruses are a phylogenetically ancient family of large DNA viruses and, depending on the type of cells in which the infection proceeds, the nature of the reproduction of the virus, the structure of the genome, molecular biological and immunological features, are divided into 3 subfamilies α, β and γ. The subfamily Alpha-herpesvirinae (α-herpes viruses) includes HSV-1, -2 and -3 (Varicella Zoster virus), which are characterized by rapid virus replication and cytopathic effects on infected cell cultures. Reproduction of α-herpesviruses occurs in various types of cells, especially epithelial, exerting a cytolytic effect.

In neurons, they cause latent persistent infection. The subfamily Beta-herpesvirinae (β-herpes viruses) is species specific. It affects various types of cells, which at the same time increase in size (cytomegaly), and can cause immunosuppressive conditions. The infection can take a generalized or latent form; persistent infection easily occurs in cell culture. The viruses of this group are characterized by slow growth in epithelial cells, exerting a “cytomegalic” , and lymphoproliferative effect. Viruses can be maintained latent in the epithelial cells of the salivary glands, tonsils, kidneys, lymphocytes, secretory glands, kidneys and other tissues. This group includes CMV, HSV-6, and HSV-7.

The subfamily Gamma-herpesviridae (γ-herpes viruses) is characterized by tropism for lymphoid cells (T- and B-lymphocytes), in which they persist for a long time with the ability to transform, causing lymphomas, sarcomas. The infectious process often stops at the prelitic stage, i.e. no formation of viral particles. Latent offspring is found in lymphoid tissue. This group includes Epstein-Barr virus and human herpesvirus type 8 which is associated with Kaposi's sarcoma (KSHV). Diseases caused by HSV type 1 and type 2 are commonly called herpes or HSV infections. Other representatives of the Herpesviridae family are called herpes viruses.

Representatives of Herpesviridae, despite many common properties characteristic of the entire family, have significant differences. Comparing the characteristics of herpes viruses, the most common causes of disease in humans, they reveal a greater number of similar signs than differences, they both have the same absolute pathogenicity, the same pathways of infection, susceptibility, a tendency to recurrent course with activation against the background of intercurrent ones, including infectious diseases. Additionally they both have lifelong persistence in the human body and the impossibility of their complete elimination. Further, they both have the ability to “escape” from factors of immune defense, and at the same time, the ability to suppress the protective functions of a person. However, there are differences: First of all, the tropism of the lesion and clinical characteristics. According to modern views of infectiology, a person, in view of his relative evolutionary youth, has not yet managed to form an equilibrium system with any of the herpes viruses, which makes them pathogenic to different degrees.

Once infected by virus, a person never actually separated with them and, as it were, carries a “unexploded bomb” inside him The rupture of a “bomb” (reactivation of a “sleeping” parasite) can occur in unfavorable situations (stress, secondary infection, hypothermia, trauma, exacerbation of a chronic disease, etc.) even after many years. This phenomenon has been observed with a latent course of herpetic and cytomegalovirus infections, chlamydia, toxoplasmosis, and HIV infection.. Today, the significance of herpesvirus infections in the formation of somatic, seemingly non-infectious diseases is largely determined.

Often with a clinically detected slow course of the infectious process that developed as a result of herpes virus infection in the prenatal period, the manifestation of its manifestations is observed not in the neonatal period, but at an older age.

It was revealed that in most children who died under the age of 14 years from various causes, the intrauterine infection and the associated immunodeficiency state were the underlying disease. And this was diagnosed only when a lethal outcome occurred. Therefore, the identification and study of latent current infection, rooted in the prenatal period, is an urgent task not only of pediatrics, but of the entire health care system. This section remains the most difficult and unexplored.

Currently, there is much evidence that persistent herpes virus infections are the etiological and pathogenetic factors in the development of chronic somatic diseases—carditis, cardiac arrhythmias, bronchial asthma, autoimmune processes, chronic and recurrent obstructive pulmonary diseases, gastric ulcer and duodenal ulcer intestines, refractory forms of chronic glomerulonephritis, diabetes mellitus, chronic fatigue syndrome, atherosclerosis with damage to the circulatory system, schizophrenia and even premature aging. Many of these diseases, even in large medical manuals, are still described as idiopathic, of unknown etiology.

There is more and more data on the role of herpes viruses in the formation of cancer. Back in 1910, the great seer I. I. Mechnikov printed an article in the Russkoe Slovo newspaper, which wrote literally the following: “One cause of cancer, of course, is in the body itself, but the other gets into it in the form of an exogenous principle, most likely a virus . . . ” In 1911, an American scientist Peyton Routh (1879-1970) discovered that chicken sarcoma (Routh sarcoma) can be transplanted not only by cells, but also by submicroscopic agents extracted from cells, i.e. caused by viruses. In 1933, Schope discovered the papilloma virus that infects rabbits in North America, in 1936. J. Bittner proved the viral origin of breast cancer in mice, in 1951.

L. Gross discovered the mouse leukemia virus, in 1964.V. Yarretom discovered the domestic cat leukemia virus, and then the monkey leukemia virus. In 1946, the Soviet virologist L.A. Zilber proposed a viral genetic theory of cancer. According to this theory, during oncogenesis, DNA of viral origin is introduced (integrated) as a fragment into the DNA of the cell and becomes an integral part of the cellular genome. This integration is the initial link in the chain of processes of further transformation of a normal cell into a cancerous one. “No matter how the tumor virus enters the human body, for a long time it does not show its presence. There is nothing surprising. He is a little pathogenic. He needs special conditions in order to be pathogenic, and while these conditions are not there, the virus is completely harmless” (L.A. Zilber). L.A. Zilber wrote: “. . . it can be considered proven that the mechanism of action of DNA or RNA viruses on a cell consists mainly in the integration of their nucleic acid with the genome of the cell, due to which hereditary changes occur in the cell, which remove the cell from subordination to the systems that regulate cell growth”.

It has now been established that the viruses involved in human cancers include DNA-containing viruses, in particular Epstein-Barr viruses and other herpes viruses. Epstein-Barr virus is present in cells in 10-20% of all cancerous tumors of the stomach. Markers of herpes simplex viruses type 1 and type 2, cytomegalovirus, Epstein-Barr virus and type 6 human herpes virus were found in the blood and bone marrow of most patients with acute leukemia during induction chemotherapy.

In a Large-Scale Study of Pathogens Causing Infectious Disease Attributed by the International Agency for Research on Cancer

In a large-scale study of infectious pathogens, classified by the International Agency of Cancer Research (International Agency for Research on Cancer Lyon, France) as carcinogenic to humans. It was revealed that out of 12.7 million newly diagnosed malignant tumors that occurred in 2008., the proportion of infectious cases was 16.1%, i.e. about 2 million. In women, cervical cancer accounted for about half the cases of infection-related outbreaks of cancer and in men, cancer of the stomach and liver infection-related was more than 80%. Malignant neoplasms of the nasopharynx, non-Hodgkin's lymphoma, and Kaposi's sarcoma were associated with family of herpes viruses (EBV) as well.

The variety of pathological processes and various diseases associated with persistent intracellular pathogens, including herpes viruses, was the reason that in 2003 the WHO Regional Office for Europe classified a group of persistent intracellular infections among the diseases that determine the future of both infectious and somatic pathologies in the human population. A few decades ago, the leading causes of childhood morbidity and mortality were acute infections. Influenza, intestinal, meningococcal infections, sepsis, pneumonia, urinary tract infections and related diseases were the main pathology that determines the life expectancy of both children and adults.

Very little time passed, and mankind armed itself with powerful anti-infective agents, among which the main ones were antibiotics and vaccines. And now the diagnoses of “cholera”, “plague”, “sepsis”, “meningitis”, and “pneumonia” are no longer the death sentence for the patient. Today, in most cases, not only is a cure possible, moreover, the disease itself caused by these infections can be prevented. Humanity began to look to the future with the hope of getting rid of infectious diseases.

Our self-confidence reached the point, that in the 60-70s of the last century, famous scientists publicly announced the end of the century of infections: “. . . the book of infections can be buried deep in the ground, they are now not scary . . . ” After all, the terrible “Spaniard” of the early twentieth century, who claimed more lives than World War I, was almost defeated. War, poliomyelitis immobility, malaria chills, tetanus and many terrible manifestations of infections go away like a nightmare. However, the “staphylococcal plague” broke out, “forgotten” poliomyelitis, diphtheria came; HIV and AIDS, Ebola, bird and swine flu, etc. Today these are herpesvirus infections, and their role in influencing human health remains to be seen. After all, we still know very little about these very common infections, and we only touched the science of the micro- and macro world surrounding us only to its closest edge.

Today we do not have generally accepted approaches to the diagnosis of diseases associated with herpesvirus infections, there are no reliable criteria, indications and the rational and adequate therapy for it. There are no sufficiently effective drugs that penetrate the cells and capable of neutralizing this group of pathogens. We hope in a series of articles on the problems of herpes virus infections to some extent to answer some of the most frequently asked questions [Yulish E.I.//Child Health. 2015. no. 3 (63).]

### The Role of Histone Deacetylase Inhibitors in the “Manifestation” of Latent Herpes Viruses and Potentiation of the Effects Of Acyclovir.

Induction of the lytic phase of EBV infection in combination with the action of an antiherpetic drug is a promising therapeutic target for the treatment of EBV-associated lymphomas and other pathologies associated with latent herpes viruses. Short chain fatty acids and other substances have been used to induce gene expression of the lytic phase of EBV in cell cultures and animals. It should be noted, however, that these studies, as a rule, have not been translated into clinical use.

The recent success of a clinical trial using arginine butyrate, a pan-HDAC inhibitor (pan-HDAC), and ganciclovir for the treatment of EBV lymphoma, has stimulated the study of the potential of several HDAC inhibitors to stimulate expression of the lytic phase of human EBV in lymphoma cells. Presented research results included the following drugs: short chain fatty acids (sodium butyrate and valproic acid); hydroxamic acids (oxaflatin, Scriptaid, suberoyl anilide hydroxamic acid, panobinostat [LBH589] and belinostat [PXD101]); benzamide MS275; cyclic tetrapeptide apicidin; and a newly discovered HDAC-leylazole inhibitor.

With the exception of suberoyl anilide hydroxamic acid and PXD101, all other HDAC inhibitors effectively sensitized EBV lymphoma cells to ganciclovir. LBH589, MS275 and croupazole were effective at nanomolar concentrations and were 104 to 110 times more effective than butyrate. The efficacy of these HDAC inhibitors makes them potentially useful as sensitizers to antiviral drugs for the treatment of EBV-associated lymphomas [Blood. 2012; 119 (4): 1008-1017].

Another article also showed that the combined treatment of infected cells with acyclovir (ACV) and valproic acid (VPA) revealed 50-250-fold potentiation of the antiviral activity of ACV. [Iran J Med Sci 2002; 27 (4): 180-187.]

### Epigalocatechin Gallate as a Histone Deacetylase Inhibitor

Since p300/CBP-mediated RelA hyperacetylation (p65) is critical for the activation of the nuclear factor KB (NF-KB), weakening p65 acetylation is a potential molecular target for preventing chronic inflammation. Inflammation plays an important role in the pathology of atherosclerosis and cancer. During a screening study to identify natural compounds with histone acetyltransferase inhibitor (HDAC) activity, epigallocatechin-3-gallate (EGCG) was identified as a novel HDACi, effective to inhibit most HDACs, including HDAC, SIRT1 and HMTase. At a dose of 100 Mmol/L, EGCG cancels p300-induced p65 acetylation in vitro and in vivo, increases the level of cytosolic IKBA and inhibits NF-KB-induced activation of tumor necrosis factor (TNFA).

It has also been shown that EGCG inhibits p65 translocation to the nucleus induced by TNFα. This confirms that hyperacetylation is critical for NF-KB transduction as well as its activity. In addition, EGCG treatment inhibited p65 acetylation and the expression of NF-KB target genes in response to various stimuli. Finally, EGCG has been shown to reduce the binding of pAC to the promoter region of the interleukin-6 gene, which emphasizes the importance of a balance between HAT and histone deacetylases in the NF-KB-mediated inflammatory signaling reaction. It is important to note that EGCG at 50 Mmol/L completely blocks the expression of cytokines induced by EBV infection, and then blocks the transformation of B-lymphocytes induced by EBV. These results show the crucial role of acetylation in the development of inflammatory diseases. [Cancer Res 2009; 69 (2): 583-92].

### Glycyrrhizic Acid

For glycyrrhizinic acid the antiviral activity is indicated against latent viruses, such as Epstein-Barr virus and Kaposi's sarcoma-associated virus. This is the only antiviral drug that has the ability to completely eliminate herpes viruses from B-lymphocytes, even when these viruses are in a latent state. Also, glycyrrhizin showed antiviral activity against cytomeglovirus, herpes viruses type 1 and 2, influenza, SARS coronavirus. [Lin J-C, Cherng J-M, Hung M-S, Baltina L a, Baltina L, Kondratenko R. Antiviral Res. 2008; 79 (1): 6-11. doi: 10.1016/j. antivira1.2008.01.160.]

### Terminology

Acylation—the introduction of the acyl residue of RCO-(acyl) into the organic compound. As a rule, by replacing the hydrogen atom, the introduction of the residue of acetic acid CH3CO— is called acetylation, benzoic C6H5CO——benzoylation, formic HCO——formylation. Depending on the atom to which the acyl residue is attached, C-acylation, N-acylation, O-acylation are isolated. Acid halides and acid anhydrides are used as acylating agents.

Alkylation—the introduction of an alkyl substituent in a molecule of an organic compound. Typical alkylating agents are alkyl halides, alkenes, epoxy compounds, and alcohols Other less frequent examples include aldehydes, ketones, esters, sulfides, and diazoalkanes. The alkylation catalysts are mineral acids, Lewis acids, and zeolites. Alkylation is widely used in the chemical and petrochemical industries.

Combinatorial synthesis—synthesis by methods of combinatorial chemistry, involves the simultaneous reaction between three or more reagents with the formation of a combinatorial synthesis product, consisting of dozens of derivatives. These derivatives are then separated chromatographically, confirm their structure and study the biological activity.

Simultaneous combinatorial modification with two modifiers—if a multifunctional molecule is used in the combinatorial synthesis reaction that has more than two groups available for modification and two modifying agents are immediately introduced into the reaction, for example, acetic anhydride and succinic anhydride. The reaction forms a mixture of acylated derivatives in different positions—acetyl-succinyl derivatives.

The combinatorial library—a set of a large number of various chemical compounds, proteins, genes or oligonucleotides, allowing you to quickly search for target genes or target proteins. For example, a kit consisting of millions of different chemicals, or a set of recombinant DNA molecules, obtained by incorporating various antibodies into the light and heavy chains of cDNAs, etc.

Histone deacetylases (Eng. Histone deacetylases, HDACs), (EC 3.5.1)—enzymes that catalyze the removal of the acetyl group of ϵ-N-acetyl-lysine of histones introduced by histone acetylases (histone acetylases, HATs) in histone H3 and K14 residues and K5, K8, K12 and K16 of histone H4, as well as the residues of some lysines of histones H2A and H2B. By modifying histones and altering chromatin conformation, histone deacetylases play an important role in the regulation of gene expression. While histone hyperacetylation by histone acetylases is usually associated with increased transcriptional activity, histone deacetylases cause hypoacetylation and, as a result, gene repression.

Hypoacetylation leads to a decrease in the gap between the nucleosome and the DNA wound on it. Tighter DNA packaging reduces its accessibility to transcription factors, which leads to transcriptional repression. Typically, histone deacetylases act as part of large complexes that, together with other proteins, inhibit chromatin activity. Substrates of histone deacetylases can be not only histones, but also some other proteins (p53, E2F, a-tubulin and MyoD). The family consists of 18 proteins belonging to 4 classes. 11 representatives belonging to I (reduced potassium dependency 3 (RPD3)-like; HDAC1, HDAC2, HDAC3, HDAC8), and II (yeast histone deacetylase class 1. Hda1. These are not to be confused with HDAC11, HDAC4, HDAC5, HDAC6, HDAC7, HDAC9 , HDAC10) and class IV (HDAC11), are called “classical” histone deacetylases, while representatives of class III are called sirtuins. Representatives of classes I and II are inhibited by trichostatin A (TCA, TSA), while representatives of other classes are insensitive to it.

### Histone Deacetylase Inhibitors (HDACi)

At the moment, there are a number of known histone deacetylase inhibitors, starting with complex compounds isolated from bacteria and fungi (TCA, tapoxin), and ending with relatively simple compounds (butyrate). Most HDACi have a three-component structure consisting of a zinc binding site, a linker, and a sequence interacting with amino acid residues at the entrance to the HDAC active site. Inhibitors of classical deacetylases function by displacing zinc ion from the active center and thus inactivating the charge-changing system. TCA has the optimal conformation for getting into the active center, having a hydroxamate group and a five-carbon linker in front of the phenyl group. TCA causes the strongest reversible effect of the known HDACis (its IC50% is in the nanomolar region). HDACi cause hyperacetylation, transcription activation, and according to some reports, it can cause active DNA demethylation.

Since HDACi slows down growth and leads to the differentiation and apoptosis of cancer cells. Active development is underway for their use in cancer therapy (vorinostat, romidepsin, proteinostat). HDACi induce apoptosis, cell cycle arrest, aging, differentiation, cell immunogenicity and inhibit angiogenesis in some cancers. The most successful examples of HDACi use are vorinostat and romidepsin in patients with refractory cutaneous and peripheral T-cell lymphoma. In accordance with the chemical structure, 4 classes of HDACi can be distinguished—hydroxamates, cyclic peptides, aliphatic acids and benzamides. Most of the information about these molecules is based on cancer research. Pan-HDACi (non-specific HDACi) are mainly hydroxamates.

Hydroxamates are represented by trichostatin A (TSA), which inhibits cell growth in lung and breast cancer and is a pan-cell HDAC inhibitor. TSA did not enter clinical practice due to adverse events including, normal cell apoptosis and DNA damage. Suberanylhydroxamic acid (SAHA) (vorinostat) is also a hydroxamate, the first FDA-approved HDACi for clinical use. Its action leads to the activation of the antiproliferative genes p21WAF1, p27 KIP1, DR5 and TNFα. It also leads to a decrease in the activity of positive growth regulators: CDK2, CDK4, cyclin D1 and cyclin D2. Currently, many molecules from the class of hydroxamates are being studied: e CBHA, LAQ-824, PXD-101, LBH-589, ITF2357, oxamflatin, ABHA, SBHA, Scriptaid, pyroxamide, SK-7041, SK-7068 and tubacin.

Recently, pan-HDACi activity against class IIa class HDAC has been questioned, but more detailed studies have revealed “true” pan-HDACi, such as pandacostat. Further prospects for pan-HDACi are complicated by the fact that they are ineffective against solid tumors (the reasons for this remain unknown). Considerable attention is currently being paid to the development of HDACi selective for certain HDAC isoforms. However, the search for new pan-HDACi continues.

The actions of pharmaceutical companies are evidence of this: for example, in September 2014, Servier and Pharmacyclists entered into an agreement on the joint development of abexinostat and other compounds. Pan-HDACi of the “new generation” appear, such as a givinostat, and clinical trials of “old” HDACi, such as panabinostat as part of mono- and combination therapy, including solid tumors, continue.

The closest prototype of the proposed composition is US patent [U.S. Pat. No. 9,334,272 B2] “Derivatives of purine and pyrimidine antiviral agents and their use as promising anticancer agents” which lists various antiherpetic derivatives of acyclovir in combination with histone deacetylase inhibitors for cancer therapy, which potentiated each other and suppressed growth cancer cells.

The disadvantage of this prototype is the absence in the composition of a combinatorial derivative of a bivalently modified epigallocatechin gallate, which even in an unmodified form is an inhibitor of all histone deacetylases. In a modified form its effective dose is 40 times higher with respect to SIRT1 and HDAC-3, in addition, it is combined with acyclovir is able to completely eliminate the pathogen both in vivo and in vitro. Also in the prototype there is no combinatorial derivative of bivalently modified glycyrrhizin, which has the ability to suppress the replication of herpes viruses in the latency phase. The prototype does not show the ability of their derivatives to cause the complete elimination of the causative agents of herpes viruses, but only an increase in the toxicity of derivatives against some cancer cells.

### DISCLOSURE OF INVENTION

The basis of the invention is the task to obtain a pharmaceutical composition intended for the elimination of herpes virus infection pathogens from tissues, including histone deacetylase inhibitors and derivatives of acyclovir, epigallocatechin gallate and glycyrrhizic acid. As well as pharmaceutically acceptable auxiliary excipients, characterized that they additionally contain a supramolecular structure based on an undivided mixture of combinatorial derivatives of epigallocatechin obtained by simultaneously modifying it with at least two covalent modifying agents.

In another embodiment of the invention, in addition to the above substances, a supramolecular structure based on an undivided mixture of combinatorial derivatives of glycerrhizin obtained by simultaneously modifying it with at least two covalent modifying agents can be added.

In this case, as covalent modifiers of epigalocatechin, you can use: combinations of succinic anhydride and monochloroacetic acid, maleic anhydride and succinic anhydride, maleic anhydride and succinic anhydride. Also, any two modifiers from the list can be used as covalent modifiers of epigalocatechin and glycerrhizin: acetic anhydride, propionic anhydride, butane anhydride, acetic-propionic anhydride, acetic-budtanic anhydride, glutaric anhydride, phthalic anhydride, cis-aconitic anhydride, trans-aconitic anhydride, citric anhydride, isoleptic anhydride, acetyl chloride, acetyl fluoride, ethylene butylohydrochloride, propionic chloride, propionic acid.

The proposed composition may also additionally contain, not limited to those presented here, but including one or more low molecular weight activators of TOLL-receptors: acridonoacetic acid, tilorone, zymosan, imidazoquinoline, imiquimod, bendazole, dipyridamole, papaverine and cholecalciferol. On the basis of this composition, various dosage forms can be prepared, including topical, oral and parenteral, and contain pharmaceutically acceptable excipients.

### BRIEF DESCRIPTION OF THE DRAWINGS

FIG. 1. Scheme for the chemical synthesis of combinatorially modified epigalocatechin gallate derivatives.

FIG. 2. Scheme for the chemical synthesis of combinatorially modified derivatives of glycyrrhizic acid.

FIG. 3. HPLC (Milichrom A-02) of the combinatorial derivative of epigalocatechin gallate (2), its octasuccinyl (1) and octaacetyl (3) derivatives and control—unmodified epigalocatechin gallate (4), solution A gradient: 0.5 M lithium perchlorate/0.1 M chloric acid, solution B: acetonitrile (B from 5% to 100%)

FIG. 4. HPLC (Milichrom A-02) of the combinatorial derivative of glycyrrhizin (2), its octasuccinyl (1) and octaacetyl (3) derivatives and control—unmodified glycyrrhizin (4), solution A gradient: 0.5 M lithium perchlorate/0.1 M perchloric acid, solution B: acetonitrile (B from 5% to 100%)

### PHARMACEUTICAL COMPOSITIONS

Various methods of preparing a patentable pharmaceutical composition (PFC) can be used. The PFC composition can be given orally or can be administered by intravascular, subcutaneous, intraperitoneal injection, in the form of an aerosol, by ocular route of administration, into the bladder, topically, and so on. For example, inhalation methods are well known in the art. The dose of the therapeutic composition will vary widely depending on the specific antiviral PFC administered, the nature of the disease, frequency of administration, route of administration, clearance of the agent used from the host, and the like. The initial dose may be higher with subsequent lower maintenance doses.

The dose can be administered once a week or once every two weeks, or divided into smaller doses and administered once or several times a day, twice a week, and so on to maintain an effective dose level. In many cases, a higher dose will be needed for oral administration than for intravenous administration, PFCs can be included in many therapeutic compositions. More specifically, the PFCs of the present invention can be incorporated into pharmaceutical compositions in combination with suitable pharmaceutically acceptable carriers or diluents, and can be incorporated into preparations in solid, semi-solid, liquid or gaseous forms, such as capsules, powders, granules, ointments, creams, foams, solutions, suppositories, injections, forms for inhalation use, gels, microspheres, lotions and aerosols.

As such, the administration of the compounds can be carried out in various ways, including oral, buccal, rectal, parenteral, intraperitoneal, intradermal, transdermal, intratracheal administration and so on. The PFCs of the invention can be distributed systemically after administration or can be localized using an implant or other composition that holds the active dose at the site of implantation. The PFCs of the present invention can be administered alone, in combination with each other, or they can be used in combination with other known compounds (e.g. clopidogrel, anti-inflammatory agents, and so on).

In pharmaceutical dosage forms, the compounds may be administered in the form of their pharmaceutically acceptable salts. The following methods and excipients are given as examples only and are in no way limiting. For oral preparations, the compounds can be used alone or in combination with suitable additives for the manufacture of tablets, powders, granules or capsules. For example, with conventional additives such as lactose, mannitol, corn starch or potato starch; with binding agents such as crystalline cellulose, cellulose derivatives, gum arabic, corn starch or gelatins; with disintegrants such as corn starch, potato starch or sodium carboxymethyl cellulose; with lubricants such as talc or magnesium stearate; and, if desired with diluents, buffering agents, moisturizing agents, preservatives and flavoring agents.

PFCs should be included in injectable compositions by dissolving, suspending or emulsifying them in an aqueous or non-aqueous solvent, such as vegetable or other similar oils, synthetic aliphatic acid glycerides, esters of higher aliphatic acids or propylene glycol; and, if desired, with conventional additives such as solubilizers, isotonic agents, suspending agents, emulsifiers, stabilizers and preservatives.

PFCs can be used in an aerosol composition for inhalation administration. The compounds of the present invention can be incorporated into suitable pressure propellants such as dichlorodifluoromethane, propane, nitrogen and the like. In addition, PFCs can be incorporated into suppositories by mixing with a variety of bases, such as emulsifying bases or water-soluble bases. The compounds of the present invention can be administered rectally using a suppository. A suppository may contain excipients, such as cocoa butter, carboax, and polyethylene glycols, which melt at body temperature but are solid at room temperature.

Standard dosage forms for oral or rectal administration, such as syrups, elixirs and suspensions, where each unit dose, for example, a teaspoon, tablespoon, tablet or suppository, may contain a predetermined amount of a composition containing one or more compounds of the present invention . Similarly, unit dosage forms for injections or intravenous administration may contain the compound of the present invention in the composition in the form of a solution in sterile water, normal saline or another pharmaceutically acceptable carrier. Implants for the sustained release of compositions are well known in the art.

Implants are made in the form of microspheres, plates, and so on with biodegradable or non-biodegradable polymers. For example, lactic and/or glycolic acid polymers form a degradable polymer that is well tolerated by the host. An implant containing the PFC according to the invention is positioned close to the focus of the pathology, so that the local concentration of the active agent is increased compared to other areas of the body. As used here, the term “Unit dosage form” refers to physically discrete units suitable for use as single doses for human and animal subjects, each unit containing a predetermined number of compounds of the present invention, which, according to calculations, is sufficient to provide the desired effect, together with a pharmaceutically an acceptable diluent, carrier or excipient.

The descriptions of the unit dosage forms of the present invention depend on the particular compound used, and the effect to be achieved, as well as the pharmacodynamics of the compound used in the host. Pharmaceutically acceptable excipients, such as fillers, adjuvants, carriers or diluents, are generally available. In addition, pharmaceutically acceptable excipients are generally available, such as pH adjusting agents and buffering agents, tonicity agents, stabilizers, wetting agents and the like.

Typical doses for systemic administration range from 0.1 μg to 1000 milligrams per kg of subject body weight per administration. A typical dose may be a single tablet for administration from two to six times a day, or one capsule or sustained release tablet for administration once a day with a proportionally higher content of the active ingredient. The effect of prolonged release may be due to the materials of which the capsule is made, dissolving at different pH values, capsules providing a slow release under the influence of osmotic pressure or any other known controlled release method.

To the professionals in the field of this art it will be clear, that dose levels may vary depending on the particular compound, the severity of the symptoms, and the subject's predisposition to side effects. Some of the specific compounds are more potent than others. Preferred doses of this compound can be readily determined by specialists who are skilled in this art in a variety of ways. The preferred method is to measure the physiological activity of PFC.

One of the methods of interest is the use of liposomes as a vehicle for delivery. Liposomes fuse with the cells of the target region and provide delivery of liposome contents to the cells. The contact of the liposomes with the cells is maintained for a time sufficient for fusion using various methods of maintaining contact, such as isolation, binding agents and the like. In one aspect of the invention, liposomes are designed to produce an aerosol for pulmonary administration. Liposomes can be made with purified proteins or peptides that mediate membrane fusion, such as Sendai virus or influenza virus and so on.

Lipids can be any useful combination of known liposome forming lipids, including cationic or zwitterionic lipids, such as phosphatidylcholine. The remaining lipids will usually be neutral or acidic lipids, such as cholesterol, phosphatidylserine, phosphatidylglycerol and the like. To obtain liposomes, the method described by Kato et al. (1991) J. Biol. Chem. 266: 3361. is utilized.

Briefly, lipids and a composition for incorporation into liposomes containing a patented composition are mixed in a suitable aqueous medium, suitably in a salt medium, where the total solids content will be in the range of about 110 wt. %. After vigorous stirring for short periods of approximately 5-60 seconds, the tube is placed in a warm water bath at approximately 25-40° C. and this cycle is repeated approximately 5-10 times.

The composition is then sonicated for a suitable period of time, typically approximately 1-10 seconds, and optionally further mixed with a vortex mixer. Then the volume is increased by adding an aqueous medium, usually increasing the volume by about 1-2 times, followed by agitation and cooling. This method allows the inlusion of supramolecular structures with high total molecular weight in liposomes.

### Compositions With Other Active Agents

For use in the methods under consideration, the PFCs of the invention can be formulated with other pharmaceutically active agents, in particular other antiviral, immunomodulatory and antimicrobial agents known in the art. Classes of drugs for the treatment of herpes virus infections and their complications are presented in standardized protocols for the treatment of these pathologies and can be combined with patented PFCs, for example, ribavirin, iododeoxyuridine, ganciclovir, valganciclovir, valaciclovir, penciclovir.

Cytokines, for example, interferon alpha, interferon gamma, interferon-beta, tumor necrosis factor alpha, interleukin 12, interferon inducers cycloferon, tilorone, other activators of TOLL receptors: zymosan, imidazoquinoline, imiquimod, inducer of completion can also be included in the PFC composition of the invention. phagocytosis of cholecalciferol. The present invention is further described by the following examples, which should not be construed as limiting the scope of the invention.

### EXAMPLE 1

### Obtaining a Combinatorial Mixture of Derivatives of Epigallocatechin Gallate (KEGG)

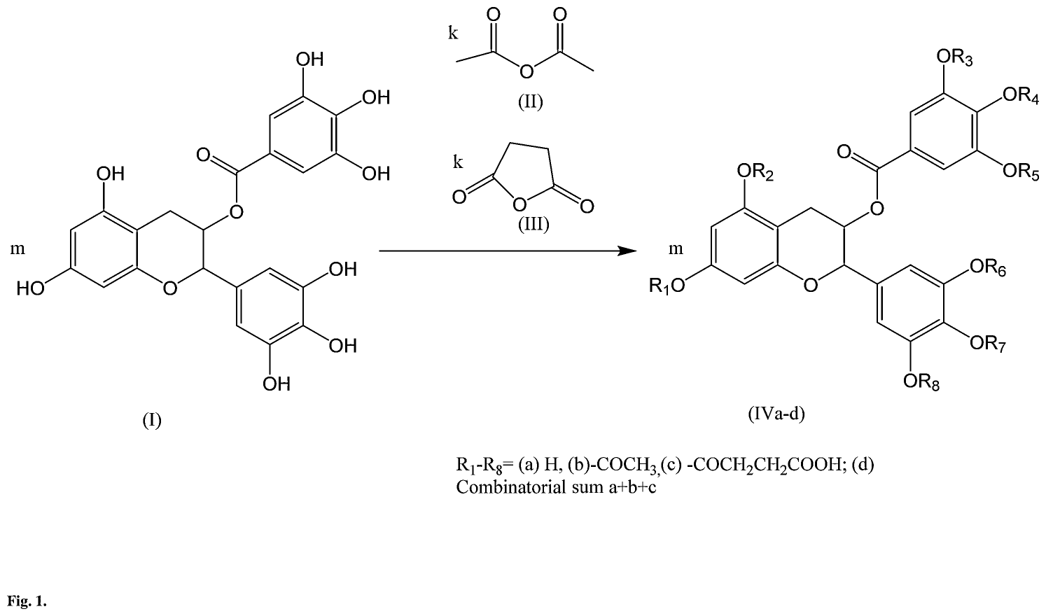

764 μM epigallocatechin gallate (I) is dissolved in 10 ml of dioxane, 2040 μM succinic anhydride (III) and 2040 μM acetic anhydride are added, the solution is stirred and heated under reflux for 20 minutes. The solution is then poured into ampoules and lyophilized to remove solvent and acetic acid. The combinatorial mixture is used to obtain pharmaceutical compositions, study the structure, and determine the biological activity. FIG. 1 shows the synthesis scheme for combinatorial derivatives of I. One initial molecule (I) contains 8 phenolic hydroxyl groups available for modification.

Calculations of the number of moles of modifiers are carried out according to the combinatorics formulas:

m=4×(3×2ⁿ⁻²−1); k=n×(2ⁿ−1), where m is the number of different derivatives of molecules in the combinatorial mixture and the number of moles (I) for the reaction; n is the number of phenolic hydroxides available for modification in structure I (n=8); k is the number of moles of each modifier.

Thus, having only one initial molecule (I) and two modifiers after combinatorial synthesis, we obtain 764 combinatorial derivatives (IV ad) with different degrees of substitution, different positions of substituents and different permutations of the modifier residues, not just in the form of a mixture, but in the form of it is difficult to separate supramolecular mixture. Modifiers—succinic anhydride or acetic anhydride can be entered both simultaneously and sequentially—or first introduce succinic anhydride, warm the mixture under reflux for 20 minutes, then add acetic anhydride and also warm the mixture for another 20 minutes.

Similarly, in this reaction, instead of succinic and/or acetic anhydrides, the following modifiers can be used: maleic anhydride, aconitic anhydride, glutaric, phthalic anhydride and acetic anhydride, formic acid ethyl ester, monochloroacetic acid, propiolactone alkylene glycol ethylene oxide , ethyl chloride, propyl chloride). C13 NMR spectra were determined on a Brucker spectrometer.

C₁₃ NMR spectra for the combinatorial derivative of epigalocatechin (IVa-d): 157.8, 94.8, 95.3, 157.2, 99.4, 24.8, 157.3, 68.6, 82.7, 130.9, 129.9, 112.0, 145.3, 144.6, 146.3, 169.0, 2-.3, 165.9 , 121.2, 108.6, 109.6, 146.1, 140.3, 133/4, 154.3, 171.1, 28.8, 29.1, 174.7

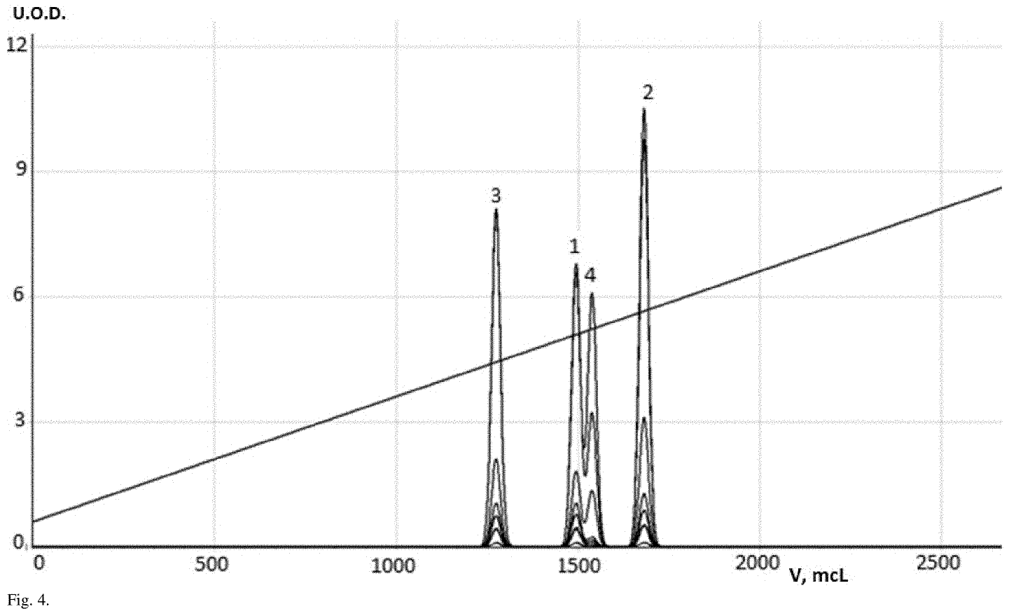

For the HPLC, a Milichrom A-02 microcolumn chromatograph in a gradient of acetonitrile (5-100%)/0.1 M perchloric acid +0.5 M lithium perchlorate was used. The combinatorial derivative in the chromatogram (FIG. 4) gave one clear broadened peak and was not divided into components, although the retention time differed from both epigallocatechin and its completely substituted derivatives. This indicated that complex supramolecular structures were formed between different combinatorial derivatives (in our case there were 764 of them), which were not separated chromatographically.

This combinatorial derivative (KEGG) also behaves similarly when separated in a thin layer (acetonitrile: water) and gives only one band, which does not coincide with any of the obtained derivatives.

Table 1 shows the screening of derivatives (I) with different ratios of modifiers as histone deacetylase inhibitors.

HAT and HDAC. Nuclear extract of HeLa cells (NE) was obtained as previously described [Yoon H G, Choi Y, Cole P A, Wong J Mol Cell Biol 2005, 25: 324-35.]. SIRT1 and HDAC-3 activity analysis was determined using a commercially available kit (Biovision Biotechnology) according to the manufacturer's instructions. SIRT1 deacetylase activity was analyzed using the SIRT1/Sir2 Deacetylase Fluorometric Assay (CycLex) kit. The table shows the data on the inhibition of histone deacetylases SIRT1 and HDAC-3 with epigalocatechin derivatives with different ratios of modifiers.

TABLE 1
The inhibitory ability against SIRT1 and
HDAC-3 from the side of supramolecular
combinatorial derivatives (I) obtained in the
reaction with different molar ratio of modifiers

| | The molar | | | | |
|---|---|---|---|---|---|
| No | ratio of reagents * | | | Kᵢ (mM) | |
| p/p | m | k1 | k2 | HDAC-3 | SIRT |
| 1 | 764 | 8160*** | 8160*** | >4 | >5 |
| 2 | -//- | 4080 | 4080 | >4 | >5 |
| 3 | -//- | 2040 | 2040 | 0.10 ± 0.02 | 0.24 ± 0.03 |
| 4 | -//- | 1020 | 1020 | 0.60 ± 0.03 | 0.80 ± 0.05 |
| 5 | -//- | 510 | 39 | >4 | >5 |
| 6 | -//- | 255 | 19 | >4 | >5 |
| 7 | -//- | 127 | 10 | >4 | >5 |
| 8 | -//- | 63 | 5 | >4 | >5 |
| 9 | -//- | 31 | 2 | >4 | >5 |
| 10 | -//- | 15 | 1 | >4 | >5 |
| 13 | -//- | 0 | 0 | >4 | >5 |
| 14 | -//- | 8160*** | 0 | >4 | >5 |
| 16 | -//- | 4080 | 0 | >4 | >5 |
| 17 | -//- | 2040 | 0 | >4 | >5 |
| 18 | -//- | 1020 | 0 | >4 | >5 |
| 19 | -//- | 510 | 0 | >4 | >5 |
| 20 | -//- | 255 | 0 | >4 | >5 |
| 21 | -//- | 127 | 0 | >4 | >5 |
| 22 | -//- | 63 | 0 | >4 | >5 |
| 23 | -//- | 31 | 0 | >4 | >5 |
| 24 | -//- | 15 | 0 | >4 | >5 |
| 25 | -//- | 0 | 1 | >4 | >5 |
| 26 | -//- | 1 | 15 | >4 | >5 |
| 27 | -//- | 0 | 31 | >4 | >5 |
| 28 | -//- | 0 | 63 | >4 | >5 |
| 29 | -//- | 0 | 127 | >4 | >5 |
| 30 | -//- | 0 | 255 | >4 | >5 |
| 31 | -//- | 0 | 510 | >4 | >5 |
| 32 | -//- | 0 | 1020 | >4 | >5 |
| 33 | -//- | 0 | 2040 | >4 | >5 |
| 34 | -//- | 0 | 4080 | >4 | >5 |
| 35 | -//- | 0 | 8160 | >4 | >5 |
| 36 | -//- | 8160*** | 1 | >4 | >5 |
| 37 | -//- | 4080 | 15 | >4 | >5 |
| 38 | -//- | 2040 | 31 | >4 | >5 |
| 39 | -//- | 1020 | 63 | >4 | >5 |
| 40 | -//- | 510 | 127 | >4 | >5 |
| 41 | -//- | 255 | 255 | >4 | >5 |
| 42 | -//- | 127 | 510 | >4 | >5 |
| 43 | -//- | 63 | 1020 | >4 | >5 |
| 44 | -//- | 31 | 2040 | >4 | >5 |
| 45 | -//- | 15 | 4080 | >4 | >5 |
| 46 | -//- | 0 | 8160 | >4 | >5 |

* m is the number of moles (1) in the combinatorial synthesis reaction; K1 is the number of moles of succinic anhydride in the reaction; K2 is the number of moles of acetic anhydride in the reaction;
** Ki (mM)—The release concentrations of the fluorescent diacetylated product of different substrate concentrations were used for Lineweaver-Burk calculations. Average data from two independent experiments.
***the maximum molar ratio at which all groups in (1) are replaced, the excess of this ratio leads to the fact that unreacted modifiers remain in the reaction medium—succinic anhydride and acetic anhydride.

As can be seen from table 1, only with the calculated ratio of components, when the maximum number of different derivatives (I) is formed, then a biologically active and effective supramolecular structure (derivative IV (a-d)) is formed. This supramolecular structure is able to inhibit both HDAC-3 and SIRT at a dose of 0.1 μM/L by 50%, which is 3 orders of magnitude less amount, than the initial dose of unmodified (I). Other derivatives either did not differ from unmodified (I) in their ability to inhibit HDAC-3 and SIRT, or were significantly less active.

This indicates that with the optimal ratio of modifiers when all possible derivatives are formed in the solution (764 variations of derivatives (I) with different permutations and arrangements in the substituents), a more complex supramolecular “quasi-lowering” structure with other properties and more than 3 orders of magnitude higher pharmacological activity is formed .

### EXAMPLE 2

### Obtaining a Fully Succinyl Epiglocatechin

10 mM epigalocatechin (I) is dissolved in 10 ml of dioxane, 80 mM succinic anhydride (III) is added, the solution is stirred and heated under reflux for 20 minutes. The solution is poured into ampoules and lyophilized.

C₁₃ NMR of octasuccinyl epigalocatechin: 174.7, 29.1, 28.8, 171.1, 149.3, 108.5, 149.6, 107.4, 155.3, 112.9, 25.0, 68.3, 82.1, 174.7, 29.1, 28.8, 133.1, 118.9, 118.9, 144.5, 133.5, 144.5, 165, 9, 123.4, 118.5, 146.0, 140.5, 146.0, 167.9

HPLC (Milichrom A-02; Gradient HClO4/LiClO4: AcCN 5-100%): 1 peak 18.0 min

### EXAMPLE 3

### Obtaining Fully Acetylated Epigalocatechin

10 mM epigalocatechin (I) is dissolved in 10 ml of dioxane, 80 mM acetic anhydride (II) is added, the solution is stirred and heated under reflux for 20 minutes. The solution is poured into ampoules and lyophilized.

C₁₃ NMR octaacetyl epigalocatechin: 20.3, 169.0, 149.3, 108.5, 20.3, 169.0, 107.4, 155.3, 112.9, 25.0, 68.3, 82.1, 133.1, 118.9, 118.9, 144.5, 133.5, 118.5, 123.4

HPLC (Milichrom A-02; Gradient HClO4/LiClO4: AcCN 5-100%): 1 peak 23.3 min

### EXAMPLE 4

### Obtaining a Combinatorial Mixture of Derivatives of Glycyrrhizin (CPG)

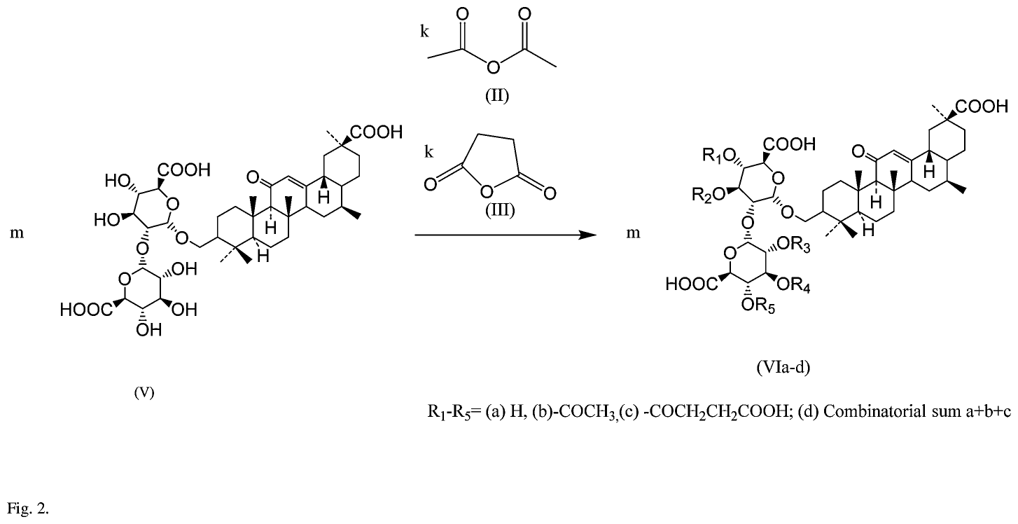

92 μM glycyrrhizin (V) is dissolved in 10 ml of dioxane, 155 μM succinic anhydride (III) and 155 μM acetic anhydride are then added. After that the solution is stirred and heated under reflux for 20 minutes. The solution is then poured into ampoules and lyophilized to remove solvent and acetic acid. The combinatorial mixture is used to obtain pharmaceutical compositions, study the structure, and determine the biological activity. FIG. 2 shows the synthesis scheme for combinatorial derivatives of V. One of the original molecule (V) contains 5 hydroxyl groups available for modification in the glycoside residue.

Calculations of the number of moles of modifiers are carried out according to the combinatorics formulas:

m=4×(3×2ⁿ⁻²−1); k=n×(2ⁿ−1), where m is the number of different derivatives of molecules in the combinatorial mixture and the number of moles (I) for the reaction; n is the number of alcohol glycoside hydroxyls available for modification in structure I (n=5); k is the number of moles of each modifier. Thus, having only one initial molecule (V) and two modifiers after combinatorial synthesis, we obtain 92 combinatorial derivatives (VI ad) with different degrees of substitution, different positions of substituents and different permutations of the modifier residues, not just in the form of a mixture, but in the form of it is difficult to separate supramolecular mixture.

Modifiers—succinic anhydride or acetic anhydride can be entered both simultaneously and sequentially—or first introduce succinic anhydride, warm the mixture under reflux for 20 minutes, then add acetic anhydride and also warm the mixture for another 20 minutes. Similarly, in this reaction, instead of succinic anhydride and/or acetic anhydride, the following can be used as modifiers: maleic anhydride, aconitic anhydride, glutaric, phthalic anhydride and acetic anhydride, formic acid ethyl ester, monochloroacetic acid, propiolactone, ethylene oxide and other low molecular weight alkylating substances (methyl chloride, ethyl chloride). C13 NMR spectra were determined on a Brucker spectrometer.

C₁₃ NMR for the combinatorial derivative of glycyrrhizin (VI ad): 174.7, 29.1, 29.5, 173.1, 172.9, 68.0, 71.6, 84.2, 82.5, 78.5, 109.8, 68.5, 68.6, 78.8, 112.1, 77.6, 173.2, 67.9, 53.1, 39.0, 29.6, 23.5, 57.2, 36.6, 17.2, 69.0, 18.0, 43.1, 40.4, 18.7, 200.8, 123.0, 158.1, 47.1, 32.4, 33.1, 15.4, 39.1, 51.5, 25.0, 43.3, 42.2, 36.2, 19.9, 182.7

For the HPLC, a Milichrom A-02 microcolumn chromatograph in a gradient of acetonitrile (5-100%)/0.1 M perchloric acid +0.5 M lithium perchlorate was used. The combinatorial derivative in the chromatogram gave one clear broadened peak and was not divided into components, although the retention time differed from both glycyrrhizin and its completely substituted derivatives. This indicated that complex supramolecular structures that were not separated chromatographically formed between different combinatorial derivatives (in our case, there were 92 of them). This combinatorial derivative (CSPG) behaves similarly when separated in a thin layer (acetonitrile: water) and gives only one band, which does not coincide with any of the obtained derivatives.

### EXAMPLE 5

### Obtaining Fully Succinylated Glycyrrhizin

10 mM glycyrrhizin (V) is dissolved in 10 ml of dioxane, 50 mM succinic anhydride (III) is added, the solution is then stirred and heated under reflux for 20 minutes. The solution is poured into ampoules and lyophilized.

C₁₃ NMR octasuccinyl glycyrrhizin: 174.7, 29.1, 29.5, 173.1, 71.6, 84.2, 82.5, 78.5, 109.8, 68.5, 68.6, 78.8, 112.1, 77.6, 173.2, 67.9, 53.1, 39.0, 29.6, 23.5, 57.2, 36.6, 17.2 , 69.0, 18.0, 43.1, 40.4, 18.7, 200.8, 123.0, 158.1, 47.1, 32.4, 33.1, 15.4, 39.1, 51.5, 25.0, 43.3, 42.2, 36.2, 19.9, 182.7

HPLC (Milichrom A-02; Gradient HClO4/LiClO4: AcCN 5-100%): 1 [symbol] 19,6

[symbol]

### EXAMPLE 6

### Obtaining Fully Acetylated Glycyrrhizin

10 mM glycyrrhizin (V) is dissolved in 10 ml of dioxane, 50 mM acetic anhydride (II) is added, the solution is then stirred and heated under reflux for 20 minutes. The solution is poured into ampoules and lyophilized.

C13 NMR octaacetyl glycyrrhizin: 172.9, 68.0, 71.6, 84.2, 82.5, 78.5, 109.8, 68.5, 68.6, 78.8, 112.1, 77.6, 173.2, 67.9, 53.1, 39.0, 29.6, 23.5, 57.2, 36.6, 17.2, 69.0, 18.0 , 43.1, 40.4, 18.7, 200.8, 123.0, 158.1, 47.1, 32.4, 33.1, 15.4, 39.1, 51.5, 25.0, 43.3, 42.2, 36.2, 19.9, 182.7

HPLC (Milichrom A-02; Gradient HClO4/LiClO4: AcCN 5-100%): 1 peak 22.1 min

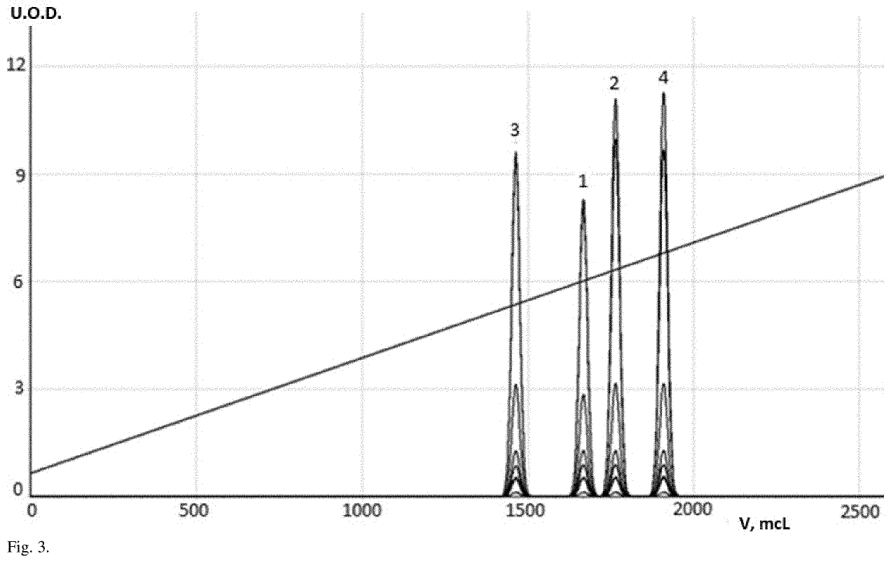

FIG. 3 shows an integrated chromatogram of 4 substances: a combinatorial derivative of epigalocatechin gallate (2), its octasuccinyl (1) and octaacetyl (3) derivatives, and control -unmodified epigalocatechin gallate (4). As can be seen from the graphs, the retention time (volume) of the derivatives differs both from the original epigalocatechin gallate and from each other, which indicates that these are different compounds. Also, peak (2) was not divided into several small fragments, nor was it possible to separate it using different conditions of thin layer chromatography, which indicates the stable nature of the formed supramolecular structure from different derivatives. Fully succinylated and fully acetylated epigalocatechins did not exhibit biological activity.

FIG. 4 shows an integrated chromatogram of 4 substances: the combinatorial derivative of glycyrrhizin (2), its octasuccinyl (1) and octaacetyl (3) derivatives, and the control is unmodified glycyrrhizin (4). As can be seen from the graphs, the retention time (volume) of the derivatives differs both from the initial glycyrrhizin and among themselves, which indicates that these are different compounds. Also, peak (2) was not divided into several small fragments, nor was it possible to separate it using different conditions of thin layer chromatography, which indicates the stable nature of the formed supramolecular structure from different derivatives. Fully succinylated and fully acetylated glycyrrhizin did not exhibit biological activity.

To check the biological (antiviral) activity of the synthesized derivatives with different ratios of components in the combinatorial synthesis reaction, the antiviral activity of the derivatives was studied by the screening method on Epstein-Barr virus models (strain X-2069b) in B-lymphoma culture plates by changing the number of copies of the virus genomes in ml PCR culture medium by detecting amplicon of LAT virus fragments (Synevo Lab). Substances were administered in 1/10 dose of the initial glycyrrhizic acid (final concentration of derivatives in the medium was 11 μm/ml), which initially had antiviral activity against Epstein-Barr virus in ED50=55 μm/ml. Cells were cultured in tablets in the environment of the Needle with the addition of donor blood plasma at a temperature of 37 0 C. The results of in vitro studies are shown in table 2.

TABLE 2
The ability of the supramolecular combinatorial
derivative of glycyrrhizin CPG to eliminate
Epstein-Barr virus from a lymphoma cell culture

| | The molar | | | The number of log |
|---|---|---|---|---|
| No | ratio of reagents * | | | copies ** virus genomes |
| p/p | M | k1 | k2 | in ml of culture fluid |
| 1 | 92 | 930*** | 930*** | >4.0 |
| 2 | -//- | 465 | 465 | >4.0 |
| 3 | -//- | 155 | 155 | 0 |
| 4 | -//- | 77 | 77 | 2.0 |
| 5 | -//- | 39 | 39 | >4.0 |
| 6 | -//- | 19 | 19 | >4.0 |
| 7 | -//- | 10 | 10 | >4.0 |
| 8 | -//- | 5 | 5 | >4.0 |
| 9 | -//- | 2 | 2 | >4.0 |
| 10 | -//- | 1 | 1 | 3.0 |
| 13 | -//- | 0 | 0 | 3.0 |
| 14 | -//- | 930*** | 0 | >4.0 |
| 16 | -//- | 465 | 0 | >4.0 |
| 17 | -//- | 155 | 0 | >4.0 |
| 18 | -//- | 77 | 0 | >4.0 |
| 19 | -//- | 39 | 0 | >4.0 |
| 20 | -//- | 19 | 0 | >4.0 |
| 21 | -//- | 10 | 0 | >4.0 |
| 22 | -//- | 5 | 0 | >4.0 |
| 23 | -//- | 2 | 0 | 3.0 |
| 24 | -//- | 1 | 0 | 3.0 |
| 25 | -//- | 0 | 1860*** | >4.0 |
| 26 | -//- | 1 | 930 | >4.0 |
| 27 | -//- | 0 | 465 | >4.0 |
| 28 | -//- | 0 | 155 | >4.0 |
| 29 | -//- | 0 | 77 | >4.0 |
| 30 | -//- | 0 | 39 | >4.0 |
| 31 | -//- | 0 | 19 | >4.0 |
| 32 | -//- | 0 | 10 | >4.0 |
| 33 | -//- | 0 | 5 | >4.0 |
| 34 | -//- | 0 | 2 | 2.0 |
| 35 | -//- | 0 | 1 | 3.0 |
| 36 | -//- | 1860*** | 1 | >4.0 |
| 37 | -//- | 930 | 1 | >4.0 |
| 38 | -//- | 465 | 2 | >4.0 |
| 39 | -//- | 155 | 5 | >4.0 |
| 40 | -//- | 77 | 10 | >4.0 |
| 41 | -//- | 39 | 19 | >4.0 |
| 42 | -//- | 19 | 39 | >4.0 |
| 43 | -//- | 10 | 77 | >4.0 |
| 44 | -//- | 5 | 155 | >4.0 |
| 45 | -//- | 2 | 465 | >4.0 |
| 46 | -//- | 1 | 930 | >4.0 |

* m is the number of moles of glycyrrhizin in the combinatorial synthesis reaction; K1 is the number of moles of succinic anhydride in the reaction; K2 is the number of moles of acetic anhydride in the reaction;
** Log means that the number in the column is the decimal logarithm of the DNA concentration, for example 3 in the column means 103 genomes/ml, the measurement error is 0.5 log/ml.
***the maximum molar ratio at which all groups in glycyrrhizin are replaced, an excess of this ratio leads to the fact that unreacted modifiers remain in the reaction medium—succinic anhydride and acetic anhydride.

As can be seen from the table, only with the calculated ratio of the components, when the maximum number of different derivatives of glycyrrhizin is formed, a biological active and effective supramolecular structure (derivative No. 3 in the table or CPGH) is formed, capable of completely eliminating the Epstein-Barr virus pathogen at a dose of 11 μm/ml from cell culture (ED100) in the absence of any cytotoxic effect on the cells. Similar effects are exerted by derivative No. 3 from Table 1 in monkey kidney cultures latently infected with viruses such as type 1 human herpes virus, type 2 human herpes virus, Zoster herpes virus, human cytomegalovirus, type 6 human herpes virus. Latent infection of the culture was caused by pretreatment of the cell culture with a subeffective concentration of acyclovir. The addition of KEGG to CPGG additionally reduced the effective concentration of CPGG by 10 times to 5 μg/ml and reduced the period of determination of the viral genome in the culture medium to 3 days.

### EXAMPLE 7

### The study of the ANTIVIRAL ACTIVITY of CPG and KEGG in an Animal Experiment (Herpes Virus Kerato-Conjunctivitis/Encephalitis in Rabbits)

As mentioned earlier, the combination of an inhibitor of histone deacetylase CPG and an antiviral agent that is effective in the latent phase of viral replication of KEGG can either eliminate the pathogen from the body or significantly reduce its load on the body. Given the role of herpes viruses in the etiology of atherosclerosis, cancer, allergic autoimmune pathologies, arthrosis, allow more successful treatment of a group of pathologies associated with herpes viruses. For studies on animal models, a 5% aqueous solution of potassium salt of an equivalent mixture of KSPG and KEGG was used.

The features of the experimental system and its level of adequacy to a natural human disease undoubtedly play a decisive role in assessing the effect of antiviral substances on the course of infection. Herpetic experimental infection is of interest due to the fact that herpetic diseases are widespread and extremely variable in clinical manifestations. Models of experimental herpes in animals are finding wider application in the study of new antiviral substances.

As you know, one of the clinical forms of systemic herpes is herpetic encephalitis, which is reproduced in guinea pigs, hamsters, rats, mice, rabbits, dogs, and monkeys. Herpetic keratoconjunctivitis in rabbits, with an average weight of 3.5 kg, was obtained by applying infectious material (herpes simplex virus type 1 strain L-2) on a scarified cornea. The animal was fixed, anesthesia of the eye was performed with dikain (instilled into the eye). Eyelids were opened, then several scratches were applied to the cornea using a syringe needle. Then the virus-containing material was introduced and, closing the eyelids, rubbed it into the cornea in circular motions.

Dose of the virus: 0.05 ml. 16 rabbits were used in the experiment, ten of them were injected with a mixture of CPG and KEGG (daily from the second day of infection −14 days at a dose of 20 mg/kg, and six-placebo (0.9% sodium chloride). After infection of the rabbits by HSV1, initiated daily monitoring of cornea, the presence of keratoconjunctivitis, encephalic disorders, and the presence of virus genomes in the blood by PCR after infection. Before infection, in all animals, amplicons of the herpes virus were absent in the blood. On day 3 after infection, HSV1 was determined in the amount of 3 log/ml in the blood of all animals in the blood. In addition, two rabbits developed encephalic manifestations—convulsive syndrome, lack of appetite. On the 4th day after infection, the experimental group of rabbits was injected into the ear vein with a mixture of CPG and KEGG at a dose of 50 μg/kg body weight, and a 0.9% sodium chloride solution was introduced into the control group.

Every day for two weeks this procedure was repeated once a day. In the experimental group, all animals survived, and the HSV1 antigen in the blood was not determined on days 13-14. In addition, in the experimental group, encephal manifestations disappeared by the 5th day of drug administration, while in the control group 2 animals died. By the 14th day of treatment, two animals died in the experimental group, while in the control group 6 died. Accordingly, the efficacy index was 83.3%, which indicates the high therapeutic efficacy of CPG/KEGG in the model of herpetic keratoconjunctivitis/encephalitis in rabbits.

In addition, the rabbits in the experimental group gained weight and all animals showed no signs of keratoconjunctivitis. The chemotherapeutic index for rabbits for the drug CPG/KEGG was 1000, which indicates the promisingness of CPG/KEGG as a highly effective antiviral drug with a wide spectrum of action and low toxicity and the ability to completely eliminate the causative agent of herpes virus.

1. A pharmaceutical composition designed to eliminate the causative agents of herpes virus infections from the tissues of a macroorganism, comprising histone deacetylase inhibitors, acyclovir derivatives, epigallocatechin gallate and glycyrrhizin, wherein it additionally contains a supramolecular structure from a combined mixture of combinatorial derivatives of gallate and epigallocatechin gallate at least two covalent modifying agents.

2. The pharmaceutical composition according to claim 1, wherein it further comprises a supramolecular structure from an undivided mixture of combinatorial derivatives of glycyrrhizin obtained by simultaneous modification of glycyrrhizin with at least two covalent modifying agents.

3. The pharmaceutical composition according to claim 1, wherein succinic anhydride and monochloroacetic acid are used as covalent modifiers.

4. The pharmaceutical composition according to claim 1, wherein maleic anhydride and succinic anhydride are used as covalent modifiers.

5. The pharmaceutical composition according to claim 1, wherein maleic anhydride and monochloroacetic acid are used as covalent modifiers.

6. The pharmaceutical composition according to claim 1, wherein any two modifiers from the list can be used as covalent modifiers: acetic anhydride, propionic anhydride, butane anhydride, acetic-propionic anhydride, acetic-butanic anhydride, glutaric anhydride, phthalic anhydride, cis-aconitic anhydride, trans-aconitic anhydride, citric anhydride, isolimonic anhydride, acetyl chloride, acetyl fluoride, propionyl chloride, butyroyl chloride, ethoxyoxalyl monochloride.

7. The pharmaceutical composition according to claim 1, wherein it further comprises not limited to those presented here, but including one or more low molecular weight activators of TOLL receptors: acridonoacetic acid, tilorone, zymosan, imidazoquinoline, imiquimod.

8. The pharmaceutical composition according to claim 1, wherein it further comprises cholecalciferol.
